(19) United States
(12) Patent Application Publication
(10) Pub. No.: US 2016/0158533 A1
(43) Pub. Date: Jun. 9, 2016

(54) Tissue Penetrating Electrode

(71) Applicant: MED-EL Elektromedizinische Geraete GmbH, Innsbruck (AT)

(72) Inventors: Claude Jolly, Innsbruck (AT); Anandhan Dhanasingh, Innsbruck (AT)

(21) Appl. No.: 15/045,299

(22) Filed: Feb. 17, 2016

Related U.S. Application Data
(63) Continuation of application No. PCT/US2014/056444, filed on Sep. 19, 2014.
(60) Provisional application No. 61/880,211, filed on Sep. 20, 2013.

(51) Int. Cl. A61N 1/05; A61B 5/00; A61N 1/36; A61B 5/04

(52) U.S. Cl. CPC A61N 1/0541; A61B 5/04001; A61B 5/6877; A61N 1/36032

(57) ABSTRACT

A tissue penetrating electrode for interfacing with a nerve includes an electrode carrier having an electrically conductive core and an electrically insulative layer disposed on the conductive core, an electrically insulative tip at its proximal end configured to penetrate into tissue, and at least one electrical contact formed from the conductive core and positioned along a radial surface of the electrode. A method of making a tissue penetrating electrode includes providing an electrode carrier having an electrically conductive core and an electrically insulative layer disposed on the conductive core. The electrode carrier has an electrically insulative tip at its proximal end configured to penetrate into tissue. The method further includes removing a portion of the insulative layer on a radial surface of the electrode carrier in order to expose the conductive core and form at least one electrical contact. A hearing device including a tissue penetrating electrode is also disclosed.

### CROSS-REFERENCE TO RELATED APPLICATIONS

The present application is a continuation application of International Application No. PCT/US2014/056444 filed Sep. 19, 2014, which claims the benefit of U.S. Provisional Patent Application No. 61/880,211 filed Sep. 20, 2013, the disclosures of which are incorporated by reference herein in their entirety.

### TECHNICAL FIELD

The present invention generally relates to electrodes for medical implants and, more particularly, the invention relates to tissue penetrating electrodes for use with hearing devices.

### BACKGROUND ART

FIG. 1 schematically shows the anatomy of a normal human ear. The ear typically transmits sounds, such as speech sounds, through the outer ear 101 to the tympanic membrane (eardrum) 102, which moves the bones of the middle ear 103 (malleus, incus, and stapes) that vibrate the oval window and round window openings of the cochlea 104. The cochlea 104 is a long narrow duct wound spirally about its axis for approximately two and a half turns. The cochlea 104 includes three chambers along its length, an upper chamber known as the scala vestibuli, a middle chamber known as the scala media, and a lower chamber known as the scala tympani. The cochlea 104 forms an upright spiraling cone with a center called the modiolus where the axons of the auditory nerve 114 reside. These axons project in one direction to the cochlear nucleus in the brainstem and they project in the other direction to the spiral ganglion cells and neural processes peripheral to the cells in the cochlea. In response to received sounds transmitted by the middle ear 103, sensory hair cells in the cochlea 104 function as transducers to convert mechanical motion and energy into electrical discharges in the auditory nerve 114. These discharges are conveyed to the cochlear nucleus and patterns of induced neural activity in the nucleus are then conveyed to other structures in the brain for further auditory processing and perception.

Hearing is impaired when there are problems in the ability to transmit sound from the external ear to the inner ear, or there are problems in the transducer function within the inner ear. To improve impaired hearing, there are several types of auditory prostheses that have been developed, such as middle ear and inner ear implants, that can restore a sense of partial or full hearing. For example, when the impairment is related to the operation of the middle ear 103, a conventional hearing aid may be used to provide acoustic stimulation to the auditory system in the form of amplified sound. When the impairment is associated with the transducer function in the cochlea 104, a cochlear implant system may be used. The cochlear implant typically includes an electrode carrier having an electrode lead and an electrode array, which is threaded into the cochlea. The electrode array usually includes multiple electrode contacts on its surface that electrically stimulate auditory nerve tissue with small currents delivered by the contacts distributed along the electrode array. These electrode contacts are typically located toward the end of the electrode carrier and are in electrical communication with an electronics module that produces an electrical stimulation signal for the implanted electrode contacts to stimulate the cochlea.

It is beneficial in some cases to interface with and stimulate the nerves directly, rather than stimulating the tissue or muscles surrounding the nerves. In order to avoid unnecessary surgical procedures that expose the nerve, or in situations where the nerve cannot be accessed by any surgical procedure, tissue penetrating electrodes may be used that can pierce through the tissue and directly reach the nerve for stimulating the nerve or for recording signals from the nerve. The current tissue penetrating electrodes typically include an electrically conductive, sharp tip at its proximal end that is used for penetrating and for the stimulation. Unfortunately, the sharp tip, which is preferred to effectively pierce the tissue, may produce a high charge density that could damage the nerves. In addition, any electrode contacts on the surface of the electrode could be dislodged during the insertion process or could increase the width of the electrode, causing further trauma of the surrounding tissue when the electrode is inserted.

### SUMMARY OF EMBODIMENTS

In accordance with one embodiment of the invention, a tissue penetrating electrode for interfacing with a nerve includes an electrode carrier having an electrically conductive core and an electrically insulative layer disposed on the conductive core, an electrically insulative tip at its proximal end configured to penetrate into tissue, and at least one electrical contact formed from the electrically conductive core and positioned along a radial surface of the electrode.

In accordance with another embodiment of the invention, a method of making a tissue penetrating electrode for interfacing with a nerve includes providing an electrode carrier having an electrically conductive core and an electrically insulative layer disposed on the conductive core. The electrode carrier has an electrically insulative tip at its proximal end configured to penetrate into tissue. The method further includes removing a portion of the insulative layer on a radial surface of the electrode carrier in order to expose the conductive core and form at least one electrical contact.

In accordance with another embodiment of the invention, an implantable hearing device for a hearing impaired patient includes an intra-scala electrode branch configured to be placed within an interior volume of a cochlea of the patient and having a plurality of electrode contacts configured to deliver a cochlear stimulation signal to adjacent neural tissue, and an intra-modiolus electrode branch having a tissue penetrating electrode configured to interface with cochlear nerve tissue within a modiolus of the patient. The tissue penetrating electrode includes an electrode carrier having an electrically conductive core and an electrically insulative layer disposed on the conductive core, and an electrically insulative tip at its proximal end configured to penetrate into tissue. The tissue penetrating electrode further includes at least one electrical contact formed from the electrically conductive core and positioned along a radial surface of the tissue penetrating electrode for delivering a modiolus stimulation signal to the cochlear nerve tissue within the modiolus of the patient.

In some embodiments, the electrical contacts may be distributed radially around the tissue penetrating electrode. The electrical contact may be a ring distributed around the radial surface of the tissue penetrating electrode. The electrical contact may be distributed along a longitudinal direction of the tissue penetrating electrode. The electrical contact may be configured to transmit a stimulation signal to the nerve or may be configured to record a signal from the nerve. The tissue penetrating electrode may include at least two electrically conductive cores, at least one electrical contact may be formed from each of the electrically conductive cores, and each electrical contact may be configured to handle a different signal. The tissue penetrating electrode may further include an insertion stopper disposed toward a distal end of the electrode that is configured to prevent the electrode from being inserted into the tissue beyond an insertion depth. The insertion stopper may further include a position marker disposed inside the stopper. The position marker may be made of a material that is detectable in radiographs. The electrode carrier may have a screw shape at its tip and along a body of the electrode carrier. The tip may have a tip attachment, in the shape of a conical-shaped pin or screw, attached to the tip. The tip may further include a drug configured to be released from the tip. The electrically insulative layer may form the tip.

### BRIEF DESCRIPTION OF THE DRAWINGS

The foregoing features of the invention will be more readily understood by reference to the following detailed description, taken with reference to the accompanying drawings, in which:

FIG. 1 schematically shows a typical human ear which includes a cochlear implant system;

FIGS. 2A-2C schematically show tissue penetrating electrodes with various electrode contact configurations according to embodiments of the present invention;

FIG. 3 schematically shows a cross-sectional view of a tissue penetrating electrode with two cores and two electrode contacts according to embodiments of the present invention;

FIGS. 4A and 4B schematically show a tissue penetrating electrode inserted into tissue and interfacing with a nerve according to embodiments of the present invention;

FIG. 5 schematically shows a tissue penetrating electrode with an insertion stopper according to embodiments of the present invention;

FIG. 6 schematically shows a tissue penetrating electrode having a screw shape along its body according to embodiments of the present invention;

FIGS. 7A and 7B schematically show a tissue penetrating electrode with a tip attachment attached to its tip according to embodiments of the present invention;

FIGS. 8A-8D schematically show various tissue penetrating electrode configurations with a drug loaded tip according to embodiments of the present invention; and

FIGS. 9A and 9B schematically show a tissue penetrating electrode as part of a double branch electrode according to embodiments of the present invention.

### DESCRIPTION OF ILLUSTRATIVE EMBODIMENTS

Various embodiments of the present invention provide a tissue penetrating electrode with an electrode carrier having an electrically conductive core and an electrically insulative layer disposed on the conductive core, an electrically insulated tip, and one or more electrode channel openings formed on its radial surface. The openings form the electrode contacts and the contacts are formed from the same conductive core that forms the electrode carrier. The benefit of a tissue penetrating electrode having this type of electrode configuration is that there are no electrical contacts on the outer surface of the electrode that could be dislodged during the tissue penetration process. In addition, since the electrode contacts are formed from the same conductive core that forms the electrode carrier, the method of manufacturing the electrode is simplified in many ways. Also, the insulative tip prevents a high charge density from forming at the tip that could damage the nerve or nerves. Details of illustrative embodiments are discussed below.

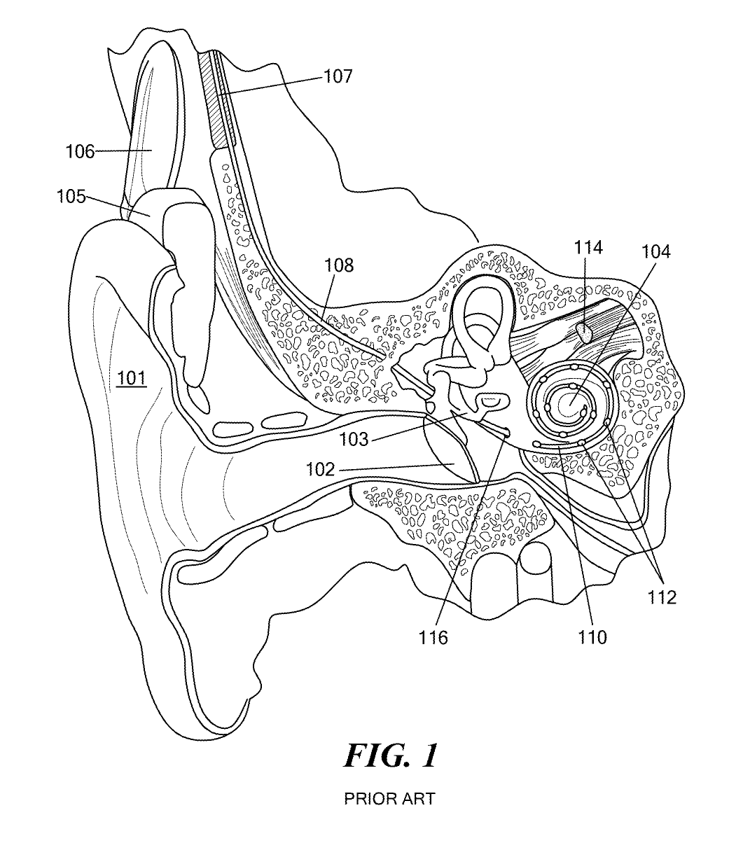

FIG. 1 shows some components of a typical cochlear implant system that may be used with embodiments of the present invention, although other hearing systems may also be used. The cochlear implant system includes an external microphone (not shown) that provides an audio signal input to an external signal processor 105 where various signal processing schemes may be implemented. The processed signal is then converted into a stimulation pattern by an external transmitter/stimulator 106, and the stimulation pattern/signal is transmitted to an implanted housing (not shown) which transmits the stimulation signal to an electrode carrier 107. The electrode carrier 107 has an electrode lead 108 and an electrode array 110 that is inserted into the cochlea 104 through an opening in the round window or a cochleostomy site 116. Typically, the electrode array 110 has multiple electrodes 112 on its surface that provide selective stimulation to the cochlea 104.

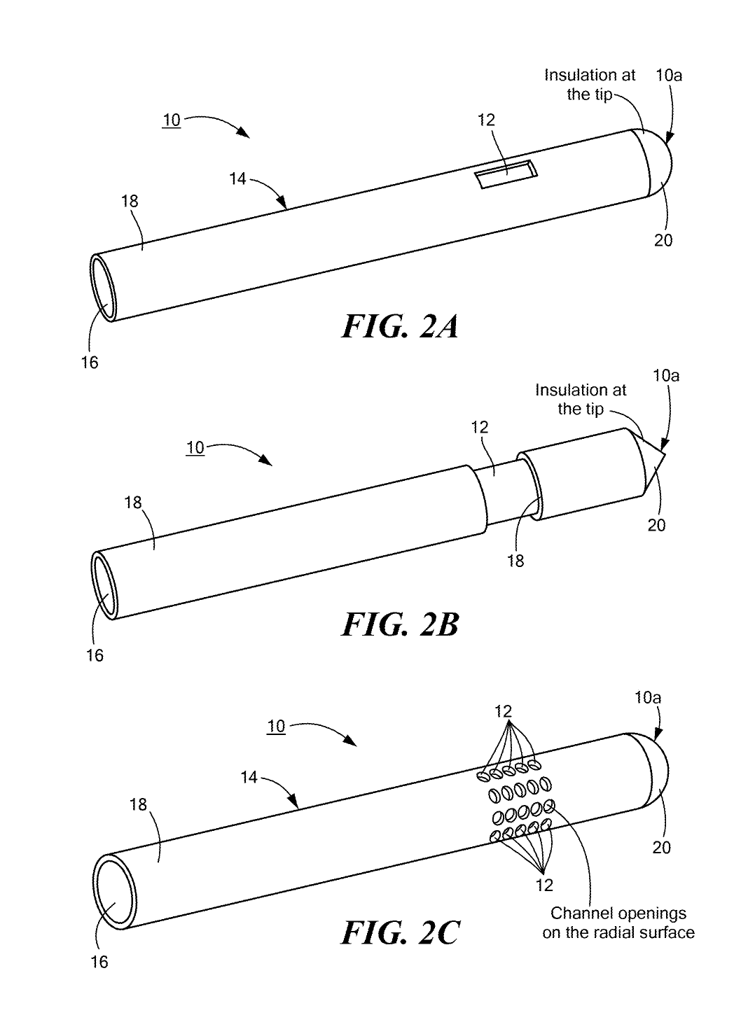

FIGS. 2A-2C schematically show tissue penetrating electrodes 10 with various electrode contact 12 configurations that may be used with hearing devices, such as cochlear implant systems. A tissue penetrating electrode 10 includes an electrode carrier 14 having an electrically conductive core 16 and an electrically insulative layer 18 disposed on the conductive core 16. The conductive core 16 may be a metal (or alloy) wire or rod, and the insulative layer 18 may be a biocompatible polymer, such as polyimide. Preferably, the electrically conductive core 16 should have an electrical resistivity of less than about 2.27×10 ⁻⁸ Ω-m (for gold) and 10.6×10 ⁻⁸ Ω-m (for platinum), at 37° C., and the electrically insulative layer 18 should have an electrical resistivity of greater than about 10×10²² Ω-m (for PTFE). Preferably, the electrically insulative layer 18 should be thick enough or have an electrical resistivity of several orders of magnitude larger than the conductive core 16 so as to prevent any current or voltage leakage through the insulative layer 18. Preferably, the conductive core 16 should have an electrical resistivity sufficiently low to allow a stimulation signal or a recording signal to be sent along the conductive core 16.

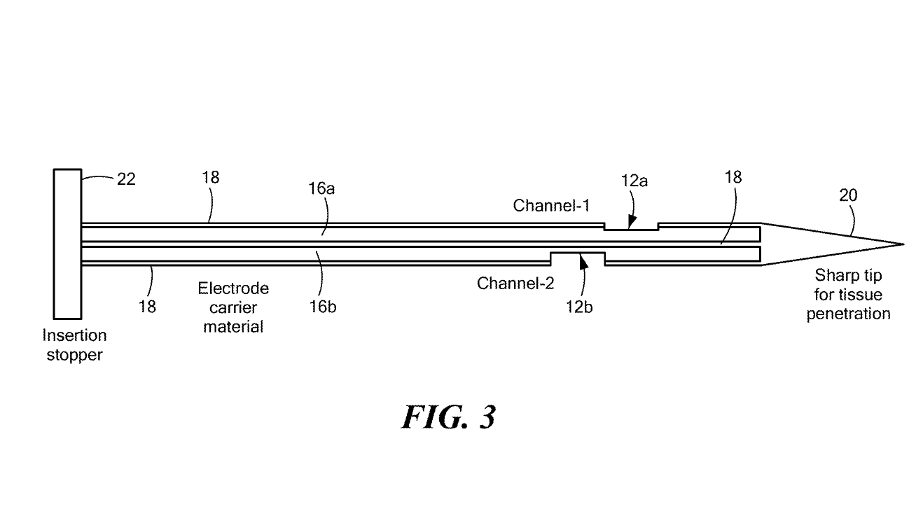

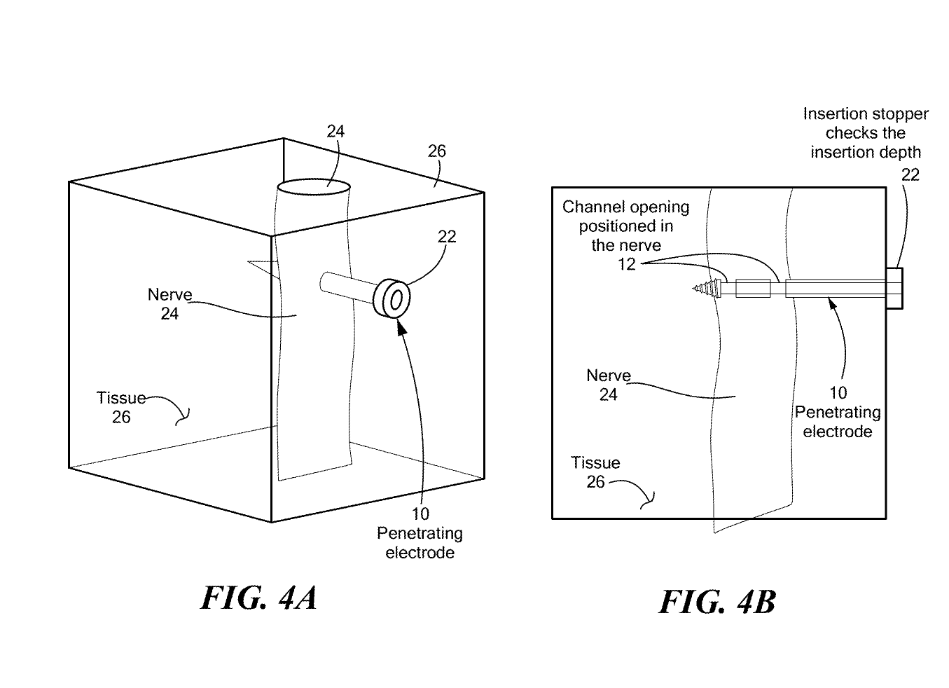

The tissue penetrating electrode 10 further includes an insulative tip 20 at its proximal end 10a that is configured to penetrate into tissue (e.g., muscle and/or bone). For example, the tip 20 may be any shape that facilitates insertion of the electrode into the tissue, e.g., a blunt tip (such as shown in FIGS. 2A and 2C), a pointed tip (such as shown in FIGS. 2B and 3), a screw-shaped tip (such as shown in FIGS. 4B and 6), etc. The conductive core 16 may be covered with the insulative layer 18 to form the insulative tip 20, or the insulative tip 20 may be formed from the same material as the insulative layer 18, as shown in FIG. 3. The benefit of this type of electrode configuration is that the insulative tip 20 prevents a high charge density from forming at the tip that could damage the nerve or nerves.

The tissue penetrating electrode 10 further includes at least one electrical contact 12 formed from the conductive core 16 and positioned along a radial surface of the electrode 10. For example, FIG. 2A shows an electrical contact 12 positioned along a longitudinal direction of the electrode, FIG. 2B shows an electrical contact 12 ring positioned around the radial surface of the electrode, and FIG. 2C shows a series of electrical contacts 12 positioned radially around the electrode. Although FIGS. 2A-2C show various electrode contact configurations, others may also be used. For example, the tissue penetrating electrode 10 may include more than one electrical contact and/or a combination of the different electrical contact configurations. For instance, the electrical contact ring (such as shown in FIG. 2B) may be used on one portion of the electrode and the series of electrical contacts (such as shown in FIG. 2C) may be used on another portion of the electrode. Similarly, in FIG. 2A, more than one electrical contact 12 may be positioned along the longitudinal direction of the electrode and/or positioned around the radial surface of the electrode and, in FIG. 2B, more than one electrical contact 12 ring may be positioned along the longitudinal direction of the electrode to form a series of rings. In FIG. 2C, a group of five electrical contacts 12 are shown along the longitudinal direction of the electrode and positioned radially around the electrode, but other numbers or configurations of electrodes may also be used.

The electrode contacts 12 are formed by removing a portion of the insulative layer 18 on the radial surface of the electrode carrier 14 in order to expose the conductive core 16. As a result, the electrical contact 12 is disposed beneath the outer surface of the electrode 10, rather than attached to its outer surface. The insulative layer 18 may be removed by any known removal process, such as laser ablation, or chemical etching. The benefit of this type of electrode configuration is that there are no electrical contacts on the outer surface of the electrode that could be dislodged during the tissue penetration process. In addition, the electrode contacts 12 are formed from the same conductive core 16 that forms the electrode carrier 14, so the method of manufacturing the electrode is simplified in some ways. Also, since the electrode contacts 12 are formed by removing the insulative layer on the conductive core 16, the overall diameter of the electrode 10 is not increased by adding electrode contacts 12 to its surface and the electrode 10 can maintain a relatively small profile, reducing the trauma on the surrounding tissue when the electrode 10 is inserted.

FIG. 3 schematically shows a cross-sectional view of a tissue penetrating electrode 10 with two conductive cores 16a, 16b. As shown, the electrically insulative layer 18 covers both conductive cores 16a, 16b and is also disposed between the two conductive cores 16a, 16b, electrically isolating each core 16a, 16b from one another. The insulative tip 20 may also be formed from the same material that forms the insulative layer 18. A portion of the insulative layer 18 is then removed from the first conductive core 16a and the second conductive core 16b in order to expose a portion of each core 16a, 16b. This configuration allows for a multi-channel tissue penetrating electrode 10 since one or more electrode contacts 12a are formed from the first conductive core 16a and one or more electrode contacts 12b are formed from the second conductive core 16b. For example, the electrode contacts 12a may be used to stimulate the nerve and the electrode contacts 12b may be used to record signals from the nerve. The benefit of this configuration is that the electrode 10 may have reduced noise since the return electrode can be included in the electrode carrier 14 itself and avoid the spread of current to the nearby nerve tissues. Alternatively, each electrode contact 12a, 12b may be used to stimulate the nerve with a different signal or different stimulation parameters.

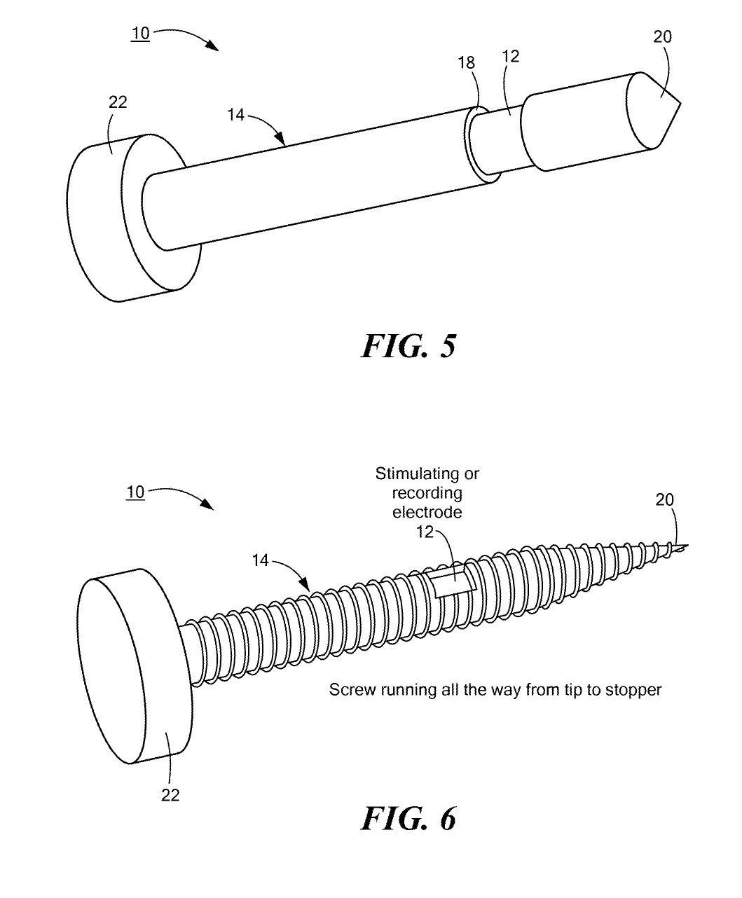

The tissue penetrating electrode 10 may also include an insertion stopper 22, such as shown in FIGS. 3 and 5, that limits the depth of penetration of a portion of the electrode 10 into the tissue. The insertion stopper 22 may be of any shape provided it is slightly bigger than the electrode 10 diameter so as to inhibit the further insertion of the electrode 10 into the tissue.

For example, as shown in FIGS. 4A and 4B, the tissue penetrating electrode 10 pierces through the tissue 26 surrounding the nerve 24 with the sharp, insulated tip 20 during the insertion process. The electrode 10 may also pierce through the nerve 24 so that the one or more electrode contacts 12 are positioned within the nerve (as shown in FIGS. 4A and 4B) or the electrode 10 may be positioned adjacent to the nerve 24 so that the one or more electrode contacts 12 are in direct contact with the nerve 24 (not shown). The insertion stopper 22 may be positioned on the electrode 10 in such a way that the depth of penetration of a portion of the electrode 10 places the electrode contacts 12 in contact with the nerve 24.

In order to facilitate the insertion of the electrode 10 into the tissue, the insulative tip 20 may be a conical-shaped pin, may be shaped like a screw, may have a blunt, rounded end, or may have any other shape that facilitates the piercing of the tissue, preferably with minimal trauma to the surrounding tissue. In addition to the tip 20, a portion of the electrode 10 may also be shaped like a screw to facilitate the insertion of the electrode 10. For example, as shown in FIG. 6, the electrode 10 may be in the shape of a screw from its tip 20 to the insertion stopper 22 along the body of the electrode carrier 14.

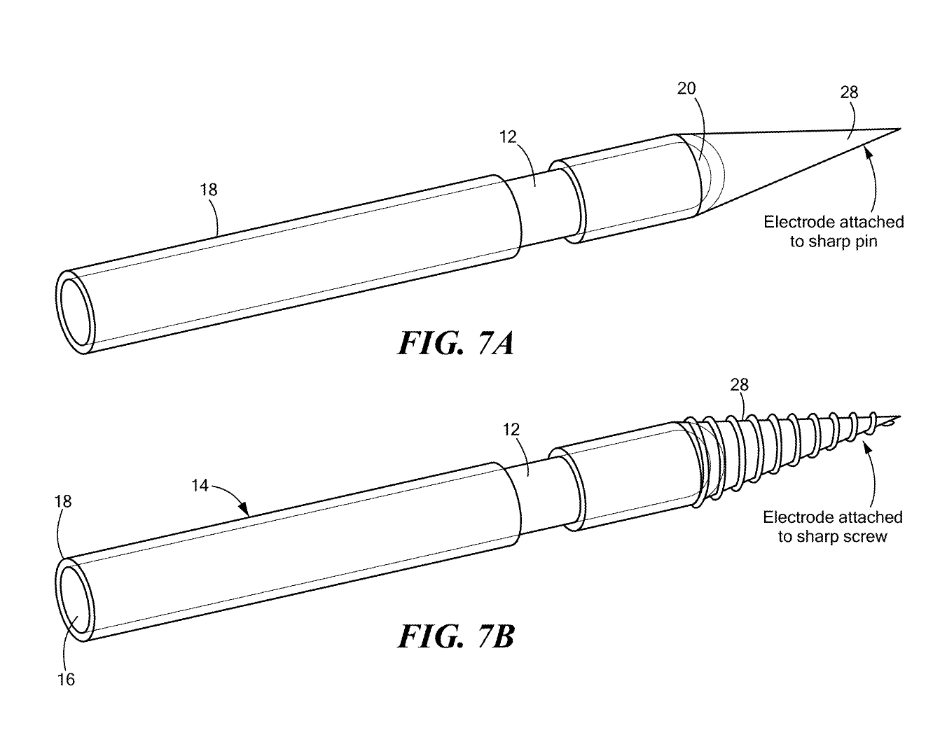

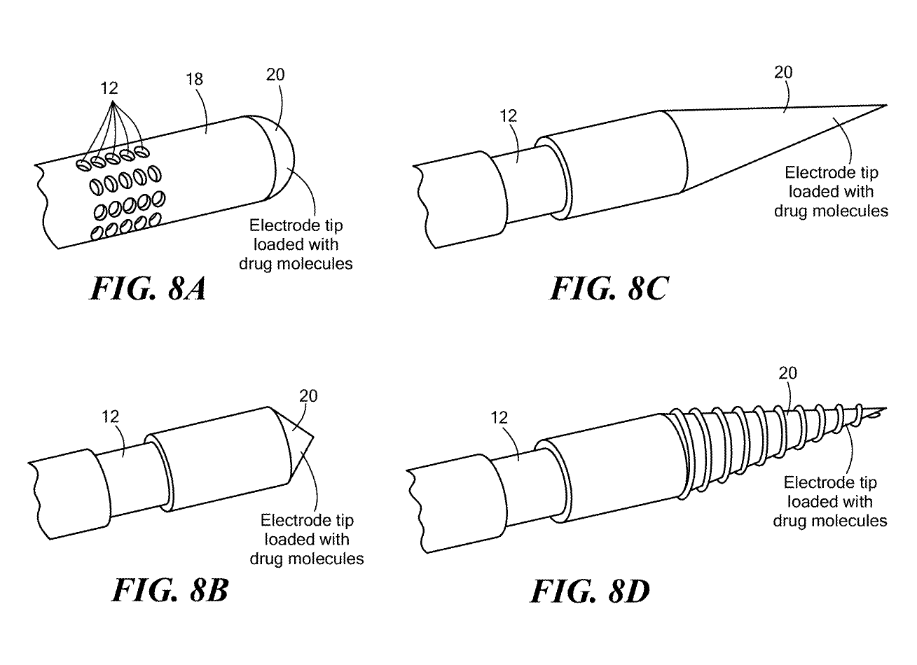

A separate tip attachment 28 may be attached to the insulative tip 20, rather than having the tip 20 in the desired shape. For example, the tip attachment 28 may be a conical-shaped, sharp pin, as shown in FIG. 7A, or may be a sharp screw, as shown in FIG. 7B. The tip attachment 28 should also be electrically insulative compared to the conductive core 16 in order to prevent a high charge density from forming at the covered tip 20 that could damage the nerve or nerves. The insulative tip 20 or tip attachment 28 may be loaded with any drug molecule for any biological purpose, such as shown in FIGS. 8A-8D. For example, the drug molecule may be selected to prevent fibrous tissue growth formation over the electrode contact 12 for better performance of the tissue penetrating electrode 10.

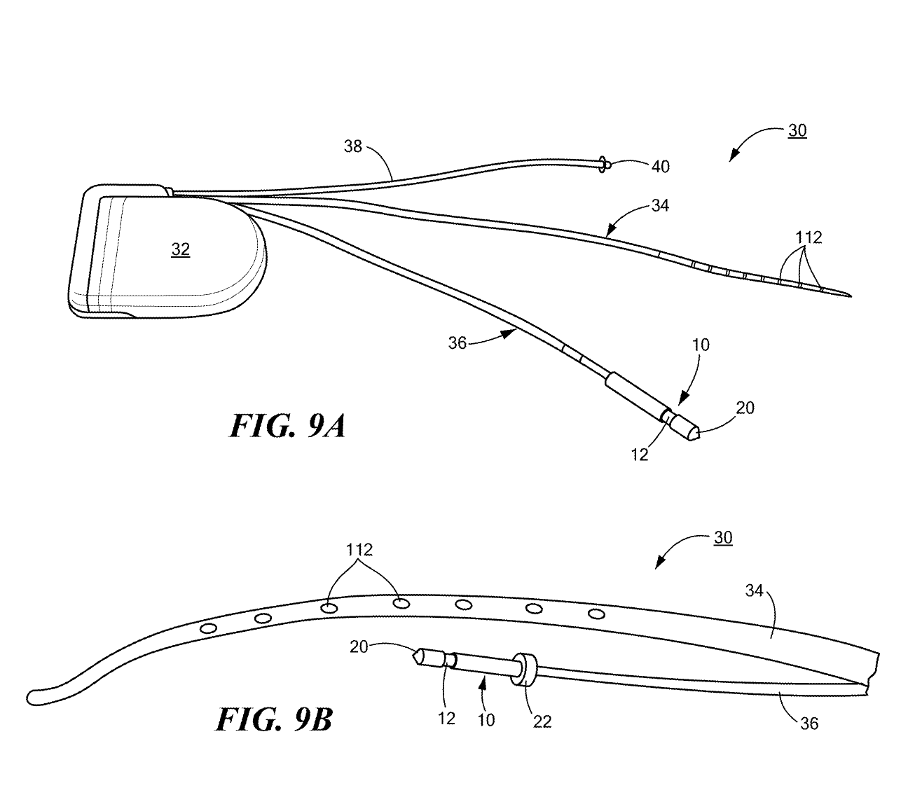

The tissue penetrating electrode 10 may also be a part of an implantable hearing device 30 that includes a double branch electrode. For example, FIGS. 9A and 9B show a stimulation electrode with two different branches, a flexible intra-scala electrode branch 34 and a flexible intra-modiolus electrode branch 36 having a tissue penetrating electrode 10 according to embodiments of the present invention. FIG. 9A shows one embodiment of the tissue penetrating electrode 10 with one electrical contact 12 ring positioned along the longitudinal direction of the electrode and FIG. 9B shows another embodiment of the tissue penetrating electrode 10 with one electrical contact 12 ring and an insertion stopper 22, although any of the previously described embodiments of the tissue penetrating electrode 10 may also be used. The hearing device 30 may include an implantable housing 32 that generates and delivers a first set of electrical stimulation signals to the intra-scala electrode branch 34. The intra-scala electrode branch 34 is configured to be positioned within an interior volume of the cochlea 104 (e.g., the scala tympani of a patient's cochlea) and immersed in cochlear fluid. The intra-scala electrode branch 34 has multiple electrode contacts 112 on its surface for delivering a cochlear stimulation signal to adjacent neural tissue. The implantable housing 32 also generates and delivers a second set of electrical stimulation signals to the intra-modiolus electrode branch 36 which is configured to penetrate through the cochlea using the tissue penetrating electrode 10 at its proximal end. As previously described, the tissue penetrating electrode 10 may include one or more electrode contacts 12 in various configurations. The electrode contacts 12 may be configured to deliver a modiolus stimulation signal to cochlear nerve tissue within the modiolus of the patient. This type of double branch electrode arrangement gives improved access to more neural tissue than for either type of electrode by itself, especially in cases of a cochlear malformation. A flexible ground branch 38, which may terminate with one or more ground electrodes 40, completes the current path for the stimulation electrodes 34, 36.

One benefit of using the implantable hearing device 30 with a double branch electrode according to embodiments of the present invention is that just a single cochleostomy can be performed at a single site, as is done for a typical cochlear implant surgery, rather than a more complex surgery entailing two cochleostomies. The intra-modiolus electrode branch 36 can also be inserted through the same posterior tympanotomy as the intra-scala electrode branch 34. Through a single cochleostomy, the thin, penetrating intra-modiolus electrode branch 36 with the tissue penetrating electrode 10 can be inserted close to or slightly through the modiolus at a specific angle of approach. In some situations, the cochleostomy may be enlarged slightly in a given direction to obtain a better angle of approach with respect to the auditory nerve in the modiolus. One advantage of the tissue penetrating electrode 10 is that it is possible to approach very close to the modiolus and nerve trunk with or without penetrating it. One or two stimulation channels in such a strategic position could also enhance system performance in a given patient. In some embodiments, the intra-modiolus electrode branch 34 may have one or more position markers on it, either in the insertion stopper 22 or along the electrode carrier 14 itself, to indicate penetration depth into the modiolus or into the cochlear nerve. The position marker may be made of a material that is detectable in radiographs so that the penetration depth can be measured and controlled during the insertion process. The insertion stopper 22 on the intra-modiolus electrode branch 36 may also be useful to prevent over-penetration of the tissue penetrating electrode 10.

Although the above discussion discloses various exemplary embodiments of the invention, it should be apparent that those skilled in the art can make various modifications that will achieve some of the advantages of these embodiments without departing from the true scope of the invention.

What is claimed is:

1. A tissue penetrating electrode for interfacing with a nerve, the electrode comprising:
an electrode carrier having an electrically conductive core and an electrically insulative layer disposed on the conductive core;
an electrically insulative tip at its proximal end configured to penetrate into tissue; and
at least one electrical contact formed from the electrically conductive core and positioned along a radial surface of the electrode.

2. The electrode of claim 1, wherein the at least one electrical contact is distributed radially around the electrode.

3. The electrode of claim 1, wherein the at least one electrical contact is a ring distributed around the radial surface of the electrode.

4. The electrode of claim 1, wherein the at least one electrical contact is distributed along a longitudinal direction of the electrode.

5. The electrode of claim 1, wherein the at least one electrical contact is configured to transmit a stimulation signal to the nerve or configured to record a signal from the nerve.

6. The electrode of claim 1, wherein the electrode includes at least two electrically conductive cores, and at least one electrical contact is formed from each of the electrically conductive cores, wherein each electrical contact is configured to handle a different signal.

7. The electrode of claim 1, further comprising an insertion stopper disposed posterior to the at least one electrode contact along the electrode, the stopper configured to prevent a portion of the electrode from being inserted into the tissue beyond an insertion depth.

8. The electrode of claim 7, wherein the insertion stopper further includes a position marker disposed inside the stopper, the position marker made of a material that is detectable in radiographs.

9. The electrode of claim 1, wherein the electrode has a screw shape at its tip and along a portion of the electrode.

10. The electrode of claim 1, wherein the tip further includes a conical-shaped pin or screw attached to the tip.

11. The electrode of claim 1, wherein the tip further includes a drug configured to be released from the tip.

12. A method of making a tissue penetrating electrode for interfacing with a nerve, the method comprising:
providing an electrode carrier having an electrically conductive core and an electrically insulative layer disposed on the conductive core, the electrode carrier having an electrically insulative tip at its proximal end configured to penetrate into tissue; and
removing a portion of the insulative layer on a radial surface of the electrode carrier in order to expose the conductive core and form at least one electrical contact.

13. The method of claim 12, wherein the at least one electrical contact is distributed radially around the electrode carrier.

14. The method of claim 12, wherein the at least one electrical contact is formed by removing a radial section of the insulative layer around the conductive core in order to form an electrical contact ring.

15. The method of claim 12, wherein the at least one electrical contact is formed by removing a section of the insulative layer along a longitudinal direction of the electrode carrier.

16. The method of claim 12, further comprising attaching an electrically insulative, conical-shaped pin or screw to the tip.

17. The method of claim 12, wherein the electrically insulative layer forms the tip.

18. The method of claim 12, further comprising providing a drug configured to be released from the tip.

19. The method of claim 12, wherein the electrode has a screw shape at its tip and along a portion of the electrode.

20. The method of claim 12, further comprising providing an insertion stopper disposed posterior to the at least one electrode contact along the electrode, the insertion stopper configured to prevent a portion of the electrode from being inserted into tissue beyond an insertion depth.

21. An implantable hearing device for a hearing impaired patient, the device comprising:
an intra-scala electrode branch configured to be placed within an interior volume of a cochlea of the patient and having a plurality of electrode contacts configured to deliver a cochlear stimulation signal to adjacent neural tissue; and
an intra-modiolus electrode branch having a tissue penetrating electrode configured to interface with cochlear nerve tissue within a modiolus of the patient, the tissue penetrating electrode including:
an electrode carrier having an electrically conductive core and an electrically insulative layer disposed on the conductive core;
an electrically insulative tip at its proximal end configured to penetrate into tissue; and
at least one electrical contact formed from the electrically conductive core and positioned along a radial surface of the tissue penetrating electrode for delivering a modiolus stimulation signal to the cochlear nerve tissue within the modiolus of the patient.
